(19) United States
(12) Patent Application Publication
(10) Pub. No.: US 2004/0243136 A1
(43) Pub. Date: Dec. 2, 2004

(54) Dual cut surgical saw blade

(76) Inventors: Parag Gupta, Portage, MI (US); Mag O'Keeffe, Cork (IE)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 10/856,597

(22) Filed: May 28, 2004

Related U.S. Application Data
(60) Provisional application No. 60/474,591, filed on May 30, 2003.

(51) Int. Cl. A61B 17/32

(52) U.S. Cl. 606/82

(57) ABSTRACT

A surgical saw blade is operatively coupled to an oscillatory power tool for oscillation about an oscillation axis (OA) and a plurality of cutting teeth are separated from one another by a clean out opening along a distal end. Each tooth presents oppositely disposed cutting edges extending transversely to the side faces and offset in opposite directions from each other. Each cutting edge has a length equal to the thickness (t) of the blade. One cutting edge protrudes a distance (de) from one side face of the blade and the other cutting edge protrudes the same distance (de) from the other side face of the blade. The offset (de) provides for cutting a groove having a width (dg) wider than the thickness (t) of the blade. Each tooth includes a V-shaped valley between the cutting edges thereof with the apex of the valley disposed on the centerline (c/l) of the tooth.

### CROSS-REFERENCES TO RELATED APPLICATIONS

This application claims the priority and benefits of provisional application Ser. No. 60/474,591 filed May 30, 2003.

### BACKGROUND OF THE INVENTION

1. Field of the Invention

The subject invention relates to a micro saw blades, and more particularly to a dual-cut micro saw surgical blade used in surgical operations for removing tissue, cartilage, and bone.

2. Background of the Related Art

Typically, surgical saw blades of different designs are used in small bone surgeries and osteotomies such as foot, oral, maxillofacial and mandibular surgery and are known in the prior art. The surgical saw blades are typically mounted on a drive unit by which they can be induced to perform an oscillating reciprocating movement to carry out, for instance, precise bone or tissue cuts. When using such surgical saw blades, it is important that the cuts are carried out precisely at the correct position and that the surrounding bone is not damaged more than necessary.

A typical prior art surgical saw blade includes a tooth pattern that typically incorporates a space between adjacent teeth, which is further defined by surfaces which lie in planes generally perpendicular to the flat surfaces of the surgical saw blade. This pattern is satisfactory in various applications wherein the teeth of the surgical saw blade exit the cut and deposit cuttings, stored in the space, outside the cut. In this mode of the surgical saw blade operation, the teeth typically do not leave the cut, whereby cuttings tend to build up in the space between the teeth thereby reducing the efficiency and speed of the surgical saw blade.

In addition, the state of the art discloses many other designs of surgical saw blades, which include crossed teeth engagement. However, such crossed surgical saw blades are not sufficiently precise and have the disadvantage, wherein the surgical saw blade becomes untrue during cutting operations, thereby providing unprecise non-perfect cut of the bone or the tissue and reducing a precise and smooth cut of wedge, thereby limiting good cut efficiency.

On some of the current micro blades on the market, the kerf has the same contour as the blade cut edge; this causes “kicking” to occur. This is caused by the cutting edge lying on the same radius as the radius of the tool rotation. All teeth engage at the same time, hence causing the blade to grab sending a force back through the hand piece to the surgeon. This sudden unpredictable movement causes inaccuracy in the cut, and requires greater control and focus from the surgeon in anticipation of the “kick”.

Another disadvantage noted in existing micro blades is the tendency of the blade to initially wander to the side rather than form a kerf. Most of the current micro blades have teeth that are oriented in an arc of constant radius, with the nature of small bone surgery where the bone is quite round having a small radius of curvature, we tend to get the a situation where, the curved blade meets the curved bone hence giving a point of contact which is extremely small leading to a tendency of the blade to wander before grabbing the bone.

In cutting, the rake angle that the cutting edge makes with the material being cut is very critical. If the angle is an acute angle, it is called a negative rake and if it is an obtuse angle, it is called a positive rake. The negative rake angle tooth is stronger but requires more cutting force. Tooth profile with positive rake angle is not as structurally strong but require less cutting force resulting in better cutting performance. A negative rake angle is disclosed in U.S. Pat. No. 3,905,374 to Winter and a positive rake angle is disclosed in later U.S. Pat. No. 5,122,142 to Pascaloff. In Pascaloff's design, the positive rake was introduced, however, half the teeth pointed in one direction whereas the other half pointed in another direction. The cutting performance improved because of positive rake but the blade did not have good control because only half of the edge on one side was engaged.

Various surgical saw blade patterns are shown in U.S. Pat. Nos. 5,306,285 to Miller et al; 5,423,845 to McDaniel; and PCT Publication No. WO 93/01751 to Kay et al.; and U.S. Pat. Nos. 6,022,353 and 6,503,253 to Fletcher et al. The U.S. Pat. No. 5,448,833 to Coon discloses a tooth pattern in a hand saw for cutting sheet rock or gypsum, but the teeth are all within the side planes of the side faces of the blade.

During the last thirty years, there has not been much progress in the micro blades. Most micro blades today still have the negative rake. The main reason for this is that positive rake reduces tooth strength and micro blades are one third or one-fourth the thickness of heavy-duty blade. So the challenge in the micro world was how to design a micro blade with a positive rake tooth, preferably with the dual cut and ensure that the tooth does not shear off while cutting.

### BRIEF SUMMARY OF THE INVENTION AND ADVANTAGES

The present invention provides a specific tooth design in a surgical saw blade for penetrating bone by being operatively coupled to an oscillatory power tool. The surgical saw blade comprises a shank having opposite side faces defining a uniform thickness with side edges and extending between a proximal end and a distal end. The proximal end has a hub defining an oscillation axis (OA) for attachment to an oscillatory power tool for driving engagement thereby. A plurality of cutting teeth along the distal end of the blade are separated from one another by a clean out opening. Each of the teeth presents oppositely disposed cutting edges extending transversely to the side faces and the cutting edges are offset in opposite directions from each other so that one cutting edge protrudes from one side of the blade and the other cutting edge protrudes from the other side of the blade for cutting a groove wider than the thickness of the blade.

This novel combination in tooth design for a surgical blade provides uniform cutting and ensures that each tooth is subjected to the same chip load. So compared to the present dual cut design in heavy-duty blade, the root of the tooth on the side of the blade in this new design will be under less stress. Not all of the teeth might be completely engaged at any one time but the maximum engagement happens close to longitudinal axis resulting in better control and less vibration. In addition, the micro dual cut has one generous radius between teeth to reduce the stress concentration due to bending of the tooth during cutting and to channel away debris, and the like. The stress concentration is not an issue in heavy-duty blade but it becomes an issue in micro blade because of reduced thickness of the blade. As a result of implementing dual cut tooth profile and ensuring equal chip load on the micro dual cut blade has resulted in a design, which out performs other blades.

### BRIEF DESCRIPTION OF THE SEVERAL VIEWS OF THE DRAWINGS

Other advantages of the present invention will be readily appreciated as the same becomes better understood by reference to the following detailed description when considered in connection with the accompanying drawings wherein:

FIG. 1 is a perspective view showing the surgical saw blade operatively coupled to an oscillatory power tool shown in phantom;

FIG. 2 is a plan view of the surgical saw blade showing on side face;

FIG. 3 is a side view taken along line 3-3 of FIG. 2 and showing one side edge of the blade;

FIG. 4 is an enlarged fragmentary view of the teeth in the circle 4 of FIG. 2;

FIG. 5 is yet a further enlarged fragmentary view of two of the teeth shown in the circle 5 of FIG. 4;

FIG. 6 is an enlarged fragmentary side view of the teeth in the circle 6 of FIG. 3; and

FIG. 7 is a plan view similar to FIG. 2 but showing the line of teeth on an arc struck about the oscillation axis.

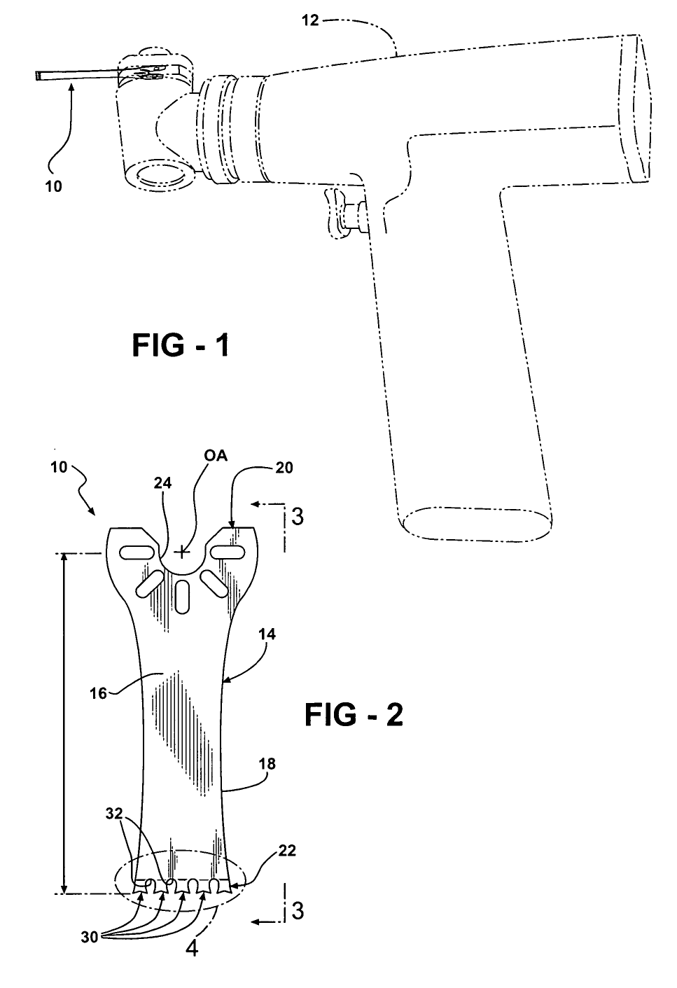

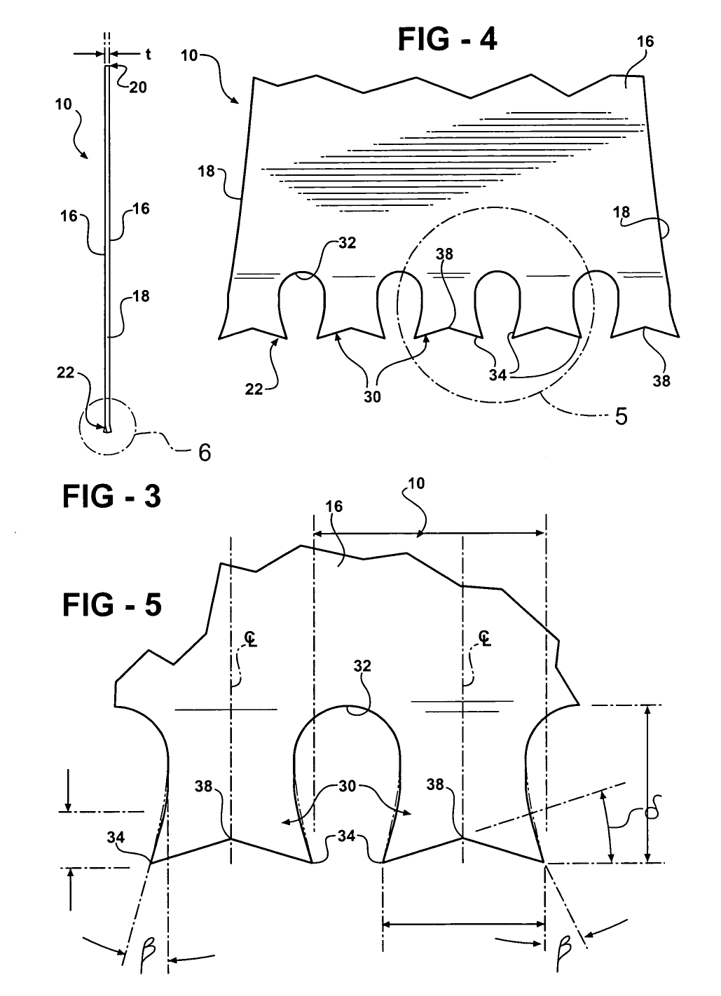

### DETAILED DESCRIPTION OF THE INVENTION

Referring to the Figures, wherein like numerals indicate like parts throughout the several views, a surgical saw blade 10 for penetrating bone is generally shown at 10. The surgical saw blade 10 is operatively coupled to an oscillatory power tool 12 for oscillation about an oscillation axis OA.

The surgical saw blade 10 comprises a shank, generally indicated at 14, having opposite side faces 16 defining a uniform thickness (t) with side edges 18 and extending between a proximal end, generally indicated at 20, and a distal end, generally indicated at 22. The blade 10 is formed from flat stock with the shank 14 being waisted between the ends 20, 22 by inwardly bowed side edges 18 thereby being narrower between the ends thereof.

The proximal end 20 is bulbous and includes a semi-circular slot 24 centered on the oscillation axis (OA) and symmetrical with the longitudinal axis of the blade 10 and a plurality of holes 26 to facilitate connection to a oscillatory power tool for driving engagement thereby.

A plurality of cutting teeth, each generally indicated at 30, are separated from one another by a clean out opening 32 along the distal end 22. The distal end 22 is straight in FIG. 2 and arcuate or curved in FIG. 7.

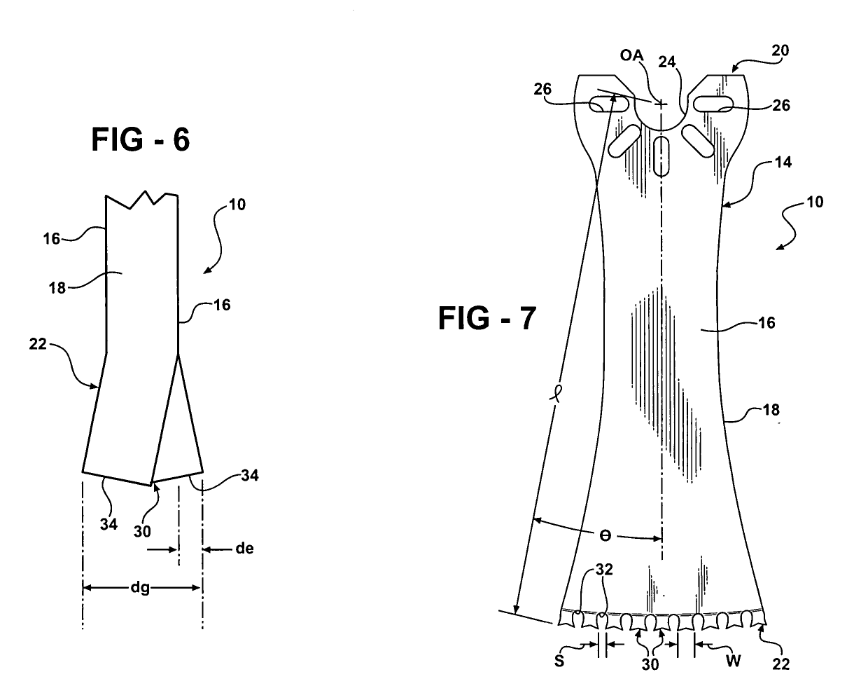

Referring to FIG. 6, each of the teeth 30 presents oppositely disposed cutting edges 34 extending transversely to the side faces 16 and offset in opposite directions from each other. Each cutting edge 34 has a length equal to the thickness (t) of the blade 10. One cutting edge 34 protrudes a distance (de) from one side face 16 of the blade 10 and the other cutting edge 34 protrudes the same distance (de) from the other side face 16 of the blade 10. The cutting edges 34 of each tooth 30 overlap in offset and intersect at an angle of less than one hundred and eighty degrees when viewed (i.e., FIG. 5) from an end of the tooth 30 along the distal end 22. The offset provides for cutting a groove having a width (dg) wider than the thickness (t) of the blade 10.

In further definition of the design of each tooth 30, as best shown in FIG. 5, each tooth 30 includes a V-shaped valley between the cutting edges 34 thereof with the apex 38 of the valley disposed on the centerline (c/l) of the tooth 30. The opposite slopes of the legs of the V-shape are at an included angle which has a complementary acute angle (δ) to one hundred and eighty degrees, i.e., one hundred eighty degrees minus the included angle of the V-shape equals a complementary acute angle (δ).

Each clean out opening 32 has a maximum width greater than the distance between the cutting edges 34 of adjacent teeth 30 for providing a relief angle (β) behind each cutting edge 34.

Referring to FIG. 7, the distance along the distal end 22 between the cutting edges 34 of each tooth 30 defines the tooth width (w) and the distance between the cutting edges 34 of adjacent teeth 30 across the clean out opening 32 defines tooth space (s) whereby the tooth width (w) plus the tooth space (s) equals the tooth pitch (p).

The arc of excursion is defined by the angle (θ). In the preferred design,

The tooth width (w) plus two times the tooth spacing (s) equals or is greater than the angle of excursion (θ) expressed in degrees divided by three hundred and sixty degrees times two pie (π) times the radius (l) of the arc of the distal end 22. This is:

w+2s=or>2lπθ/360

It has been discovered preferable for the ratio of tooth width (w) divided by tooth spacing (s) to be between zero (0) and three (3). By equation substitution, it is preferable that the tooth pitch (w+s) divided by the tooth width (w) be between one (1) and four (4).

Obviously, many modifications and variations of the present invention are possible in light of the above teachings. The invention may be practiced otherwise than as specifically described within the scope of the appended claims. In addition, the reference numerals in the claims are merely for convenience and are not to be read in any way as limiting.

What is claimed is:

1. A surgical saw blade for penetrating bone by being operatively coupled to an oscillatory power tool, the surgical saw blade comprising:
a shank having opposite side faces defining a uniform thickness (t) with side edges and extending between a proximal end and a distal end;
said proximal end having a hub defining an oscillation axis (OA) for attachment to an oscillatory power tool for driving engagement thereby;
a plurality of cutting teeth separated from one another by a clean out opening along said distal end,
each of said teeth presenting oppositely disposed cutting edges extending transversely to said side faces, said cutting edges being offset (de) in opposite directions from each other so that one cutting edge protrudes (de) from one side face of said blade and the other cutting edge protrudes (de) from the other side face of said blade for cutting a groove wider (dg) than said thickness (t) of said blade.

2. A surgical saw blade as set forth in claim 1 wherein each tooth includes a valley between said cutting edges thereof.

3. A surgical saw blade as set forth in claim 2 wherein said valley is V-shaped with the apex of the valley disposed on the centerline of said tooth.

4. A surgical saw blade as set forth in claim 3 wherein each cutting edge has a length equal to said thickness (t) of said blade.

5. A surgical saw blade as set forth in claim 4 wherein the cutting edges of each tooth overlap in offset and intersect at an angle of less than one hundred and eighty degrees when viewed from an end of said tooth along said distal end.

6. A surgical saw blade as set forth in claim 5 wherein said clean out opening has a maximum width greater than the distance between the cutting edges of adjacent teeth for providing a relief behind each cutting edge.

7. A surgical saw blade as set forth in claim 6 wherein the distance between said cutting edges of each tooth defines the tooth width (w) and the distance between the cutting edges of adjacent teeth across said clean out opening defines tooth space (s) whereby the tooth width (w) plus the tooth space (s) equals the tooth pitch (p).

8. A surgical saw blade as set forth in claim 7 wherein said tooth pitch (p) divided by the tooth width (w) is between one and four.

9. A surgical saw blade as set forth in claim 8 wherein said shank is wasted between said ends thereby being narrower between said ends hereof.
